(19) United States
(12) Patent Application Publication
(10) Pub. No.: US 2025/0125019 A1
(43) Pub. Date: Apr. 17, 2025

(54) GENERATIVE MODELLING OF MOLECULAR STRUCTURES

(71) Applicant: INTERNATIONAL BUSINESS MACHINES CORPORATION, ARMONK, NY (US)

(72) Inventors: Elise Jennings, Dublin (IE); AKIHIRO KISHIMOTO, Setagaya (JP); Oznur Alkan, Clonsilla (IE); Dmitry Zubarev, San Jose, CA (US); Seiji Takeda, Minato-ku (JP); Elizabeth Daly, Dublin (IE)

(21) Appl. No.: 18/539,942

(22) Filed: Dec. 14, 2023

(30) Foreign Application Priority Data
Oct. 11, 2023 (GB) 2315587.2

(51) Int. Cl. G16C 20/70; G16C 20/50

(52) U.S. Cl. CPC G16C 20/70; G16C 20/50

(57) ABSTRACT

A method, computer program product, and computer system for generative modelling of molecular structures for chemical applications. The method includes providing labelled training data for training a generative model over a defined feature space, where the labelled training data includes representations of molecular structures and property values for each molecular structure, and the generative model outputs generated candidate molecular structures with target properties. The method includes receiving evaluations of generated candidate molecular structure outputs from the generative model with the evaluations providing feature representations of candidates with evaluation labels. The method generates or updates decision boundary rules based on the evaluations and applies the decision boundary rules to update the labelled training data.

### BACKGROUND

The present invention relates to generative modelling of molecular structures, and more specifically, to generative modelling with learned decision boundaries for chemical applications.

Discovering new molecules has been an important subject with applications to many industrial domains where chemistry is involved, such as pharmaceutical, chemical, food and material industries.

Efficiently discovering new molecules which meet specific objectives (for example, nitrogen fixation, specific toxicity for semiconductors) is a huge challenge due to a large number of possible molecules which may exist in the real world.

In practice, trial and error experiments designing possible new structures, synthesizing them and evaluating their effectiveness, are not sustainable given the human effort required.

Artificial material discovery platforms aim to accelerate material discovery by providing chemists with artificial intelligence technologies embedded in the workflow, such as molecular structure generation. These approaches have critical expert-in-the-loop elements to assess quality of the molecular structure generation. As yet there is no method to generate an explicit representation of this human knowledge in the form of decision boundaries.

There is currently a low rate of acceptance of generative model candidates. Given the expected number of outputs ˜10\hat{ }6 for future campaigns, experiments to validate these outputs, as well as human input on each would be incredibly costly.

### SUMMARY

According to an aspect of the present invention there is provided a computer-implemented method for generative modelling of molecular structures for chemical applications, the method comprising: providing labelled training data for training a generative model over a defined feature space, wherein the labelled training data includes representations of molecular structures and property values for each molecular structure, and wherein the generative model outputs generated candidate molecular structures with target properties; receiving evaluations of generated candidate molecular structure outputs from the generative model, wherein the evaluations provide feature representations of candidates with evaluation labels; generating or updating decision boundary rules based on the evaluations; and applying the decision boundary rules to update the labelled training data.

According to another aspect of the present invention there is provided a system for generative modelling of molecular structures for chemical applications, comprising: a processor and a memory configured to provide computer program instructions to the processor to execute the function of the components: a training input component for providing labelled training data for training a generative model over a defined feature space, wherein the labelled training data includes representations of molecular structures and property values for each molecular structure, and wherein the generative model outputs generated candidate molecular structures with target properties; an evaluation component for receiving evaluations of generated candidate molecular structure outputs from the generative model, wherein the evaluations provide feature representations of candidates with evaluation labels; a decision boundary rule component for generating or updating decision boundary rules based on the evaluations; and a training data update component for applying the decision boundary rules to update the labelled training data.

According to a further aspect of the present invention there is provided a computer program product for generative modelling of molecular structures for chemical applications, the computer program product comprising a computer readable storage medium having program instructions embodied therewith, the program instructions executable by a processor to cause the processor to: provide labelled training data for training a generative model over a defined feature space, wherein the labelled training data includes representations of molecular structures and property values for each molecular structure, and wherein the generative model outputs generated candidate molecular structures with target properties; receive evaluations of generated candidate molecular structure outputs from the generative model, wherein the evaluations provide feature representations of candidates with evaluation labels; generate or update decision boundary rules based on the evaluations; and apply the decision boundary rules to update the labelled training data.

The computer readable storage medium may be a non-transitory computer readable storage medium and the computer readable program code may be executable by a processing circuit.

### BRIEF DESCRIPTION OF THE DRAWINGS

Embodiments of the present invention will now be described, by way of example only, with reference to the accompanying drawings:

FIG. 1 is a flow diagram of an example embodiment of a method in accordance with embodiments of the present invention;

FIG. 2 is a flow diagram of an example embodiment of an aspect of a method in accordance with embodiments of the present invention;

FIG. 3 is a flow diagram of an example embodiment of another aspect of a method in accordance with embodiments of the present invention;

FIGS. 4A and 4B are schematic flow diagrams of a first example embodiment of a method and system in accordance with embodiments of the present invention;

FIGS. 5A and 5B are schematic flow diagrams of second and third example embodiments of a method and system in accordance with embodiments of the present invention;

FIG. 6 is a block diagram of an example embodiments of a system in accordance with embodiments of the present invention; and

FIG. 7 is a block diagram of an example embodiment of a computing environment for the execution of at least some of the computer code involved in performing the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numbers may be repeated among the figures to indicate corresponding or analogous features.

### DETAILED DESCRIPTION

Embodiments of a method, system, and computer program product are provided which use learned decision boundaries based on evaluations of molecular structure generation model outputs to directly modify the training data for subsequent training of the model. Optionally, the method and system directly incorporate constraints for subsequent training of the generative model. The evaluations may be expert-in-the loop provided labels or predicted property drift labels. The described disclosure may also learn the predicted property drift decision boundaries and modifies the training data for the generative model to reflect a drift tolerance level.

The generative modelling of molecular structures for chemical applications is an improvement in the technical field of chemical modelling generally and more particularly in the technical field of discovering new molecules having properties suitable for chemical and material science applications. Examples of such modelling may include polymer material discovery for applications such as chip design including, for example, fan-out wafer-level packaging. This application includes resists for semiconductors and materials for the packaging, and other portions of resist development. The learned decision boundaries calculated in by the described method can be used to limit the feature space under consideration and target areas in feature space of high relevance, thus improving the efficiency of molecule generation.

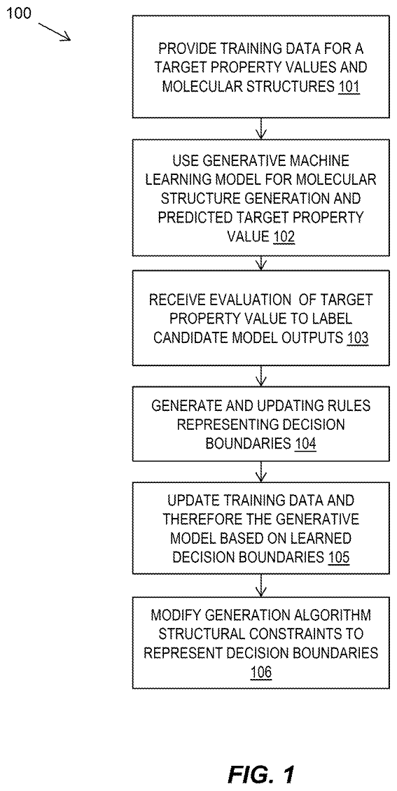

Referring to FIG. 1, a flow diagram shows an example embodiment of the described method. The described method may be provided in relation to an existing generative modelling system.

The method includes providing 101 labelled training data to a generative model for molecular structures, the molecular structures being generated for chemical applications. Generative modelling is a machine learning model or customized algorithm which takes as input labelled training data in the form of molecular structure representations and property values for each molecular structure. This model is then trained over a defined feature space. The molecular structures may be represented using a string or graph, such as SMILES (Simplified Molecular Input Line Entry System), molecular fingerprint, or substructure key representations. The training data may also include feature vectors; alternatively, the feature vectors may be generated from the molecular structure representations.

The term “molecular structure” is intended to include molecular structures representing entire molecules or substructures of a molecule, for example, core substructures that determine its chemical property.

The method may use 102 the generative model to generate candidate molecular structures which satisfy certain constraints and to predict property values for each generated molecular structure. Scaffold candidates are core substructures of a molecule which determine its chemical property and may be handled by the generative model in a similar manner to entire molecular structures. Scaffold candidates may be represented as a SMILES string or a graph structure.

The method may receive 103 evaluations of the target property value to label candidate generated molecular structures with evaluation labels to provide a feature representation of candidates with labels. The evaluations may create feature representations of the candidate molecular structures with labels.

In one embodiment, the evaluation may take molecular structures representing entire molecules with the evaluation carried out by an expert-in-the-loop or subject matter expert (SME) using ontological feature representation with labels of the candidate structure to accept/reject.

In another embodiment, the evaluation may take a scaffold candidate or an entire molecular candidate from the generative model with an associated predicted property. The candidate's actual property is measured via real or virtual experiments or simulations in order to generate an evaluation label as instances of tolerance of predicted property drift.

The method may generate 104 and update rules representing decision boundaries. A decision boundary is the term used in binary classification problems, where a decision boundary represents a hypersurface that partitions the underlying vector space into two sets, one for each class. The classifier will classify all the points on one side of the decision boundary as belonging to one class and all those on the other side as belonging to the other class. In general, for multiple classes, the decision boundary is the boundary between different classes or decision regions. The method may generate or update Boolean rules representing a decision boundary based on the target chemical property value and molecular structure and the received evaluation labels.

Generating 104 decision boundary rules may include preparing feature values for constructing conditions for the decision boundary rules including: preparing a class function for each class in an ontology diagram, where the class function checks whether a molecule in question belongs to that class or not; and preparing a list of elements where each element consists of a molecule, a label, and a set of the values calculated by the class functions.

The method may update 105 the training data to represent the decision boundaries represented as Boolean rules. The method may modify 106 the generation algorithm's structural constraints in the generational model to represent the decision boundaries represented as Boolean rules. Modifying training data may use chemical similarity measures in the feature space to represent learned decision boundaries.

Given generated chemical structures, the method automatically learns decision boundaries which generates an explicit knowledge representation in the form of a decision boundary for a chemical domain. The method also uses a given ontological or chemical space feature representation to represent an expert-in-the-loop decision or predicted property drift decision space. The method modifies the training data using concepts of chemical similarity for subsequent generative model training. The method may also directly provide the learned decision boundaries as constraints to the generative model. The method may provide a cooperative interaction system between the machine learning model and a subject matter expert (SME) as opposed to adversarial interactions.

The method automatically uses learned decision boundaries to modify training data for molecular generation.

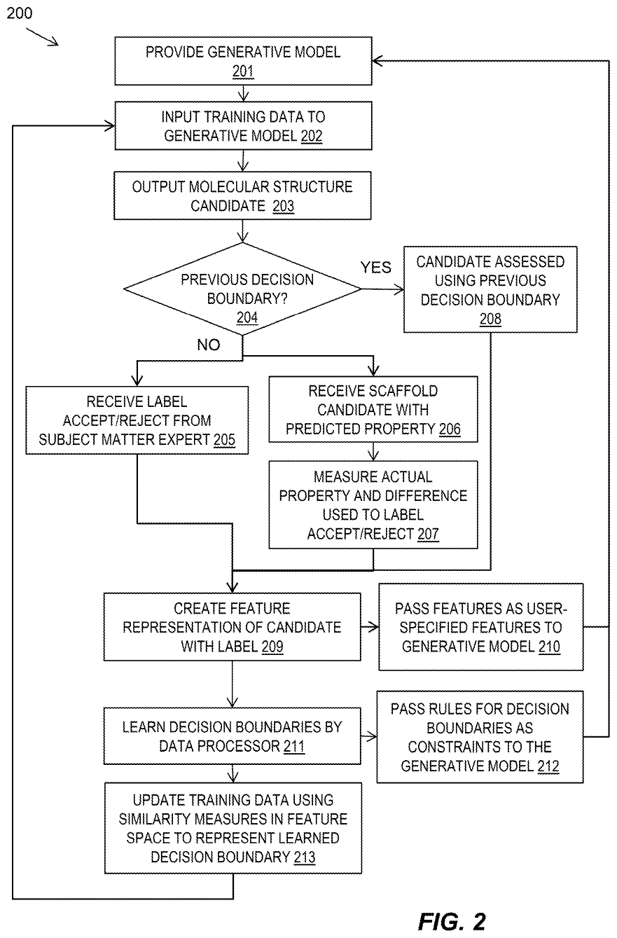

Referring to FIG. 2, a flow diagram 200 shows an example embodiment of the overall described method. The method may provide 201 a generative model for molecular structure generation and its predicted target property value. The method inputs 202 training data into the generative model. This includes inputting training data in the form of a target chemical property value and a molecular structure representation, e.g., molecule represented as SMILES. The machine learning model outputs 203 a candidate molecular structure that satisfies certain constraints and to predict property values of the structure.

The method determines 204 if there are previous decision boundary rules for the molecular structure. If there are previous decision boundary rules, the candidate molecular structure is assessed 208 using the previous decision boundary rules. If there are no previous decision boundary rules, the method may evaluate the candidate molecular structures by, in one embodiment, receiving 205 accept/reject labels from a subject matter expert (SME) as an expert-in-the-loop using an ontological feature representation.

Alternatively, the method may evaluate the candidate molecular structures by receiving 206 a scaffold candidate from the generative model with an associated predicted property. The candidate's actual property is measured 207 via real or virtual experiments or simulations in order to generate an accept/reject label as instances of predicted property drift.

Following the parallel options of either using 208 previous decision boundary rules, receiving 205 SME input, and receiving 206 and measuring 207 predicted property properties and their drift, the method may create 209 feature representations of the candidate molecular structure with labels. The features may be passed 210 as user-specified features to the generative model.

The method may learn 211 or update rules representing decision boundaries based on the target chemical property value and molecular structure. The method may pass 212 the decision boundary rules as constraints to the generative model.

The method may update 213 the training data to represent the decision boundaries represented as Boolean rules.

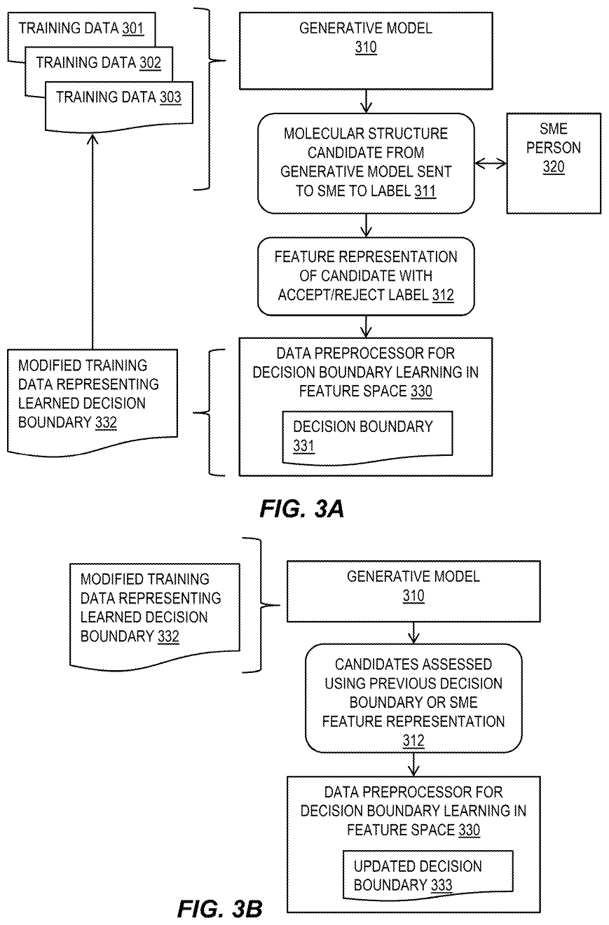

Referring to FIGS. 3A and 3B, a first embodiment is described using a schematic flow diagram of the method and system components. The first embodiment uses expert-in-the-loop learned decision boundaries.

The method may input training data 301-303 into a generative model 310. The input data 301-303 may be SMILE string representations and features, e.g., atom counts, substructure counts, InChiKeys (International Chemical Identifiers) or Morgan Fingerprint float features.

The generative model 310 may provide feature encoding, property prediction, feature search, structure generation and may output molecular structure candidates 311 that are sent to a subject matter expert 320 to provide an evaluation label, for example, as: Yes, No, or Uncertain.

Feature representations 312 are provided of the candidates from a constitutional ontology together with the evaluation label from the SME 320.

A data preprocessor 330 is used to process the feature representations of candidates and SME labels to learn the decision boundaries 331 in the feature space.

Modified training data 332 is provided using chemical similarity measures in the feature space to represent learned decision boundary, for example, rdkit.DataStructs.FingerprintSimilarity( ).

Referring to FIG. 3B, the generative model 310 is trained again using the modified training data 332 and outputs more candidates 312. New candidates breaking constraints are not generated. Candidates 312 may be assessed using previous decision boundaries, where available, or using input from the subject matter exert to output an ontology feature representation for further processing by the data preprocessor 330 to update decision boundary rules 333.

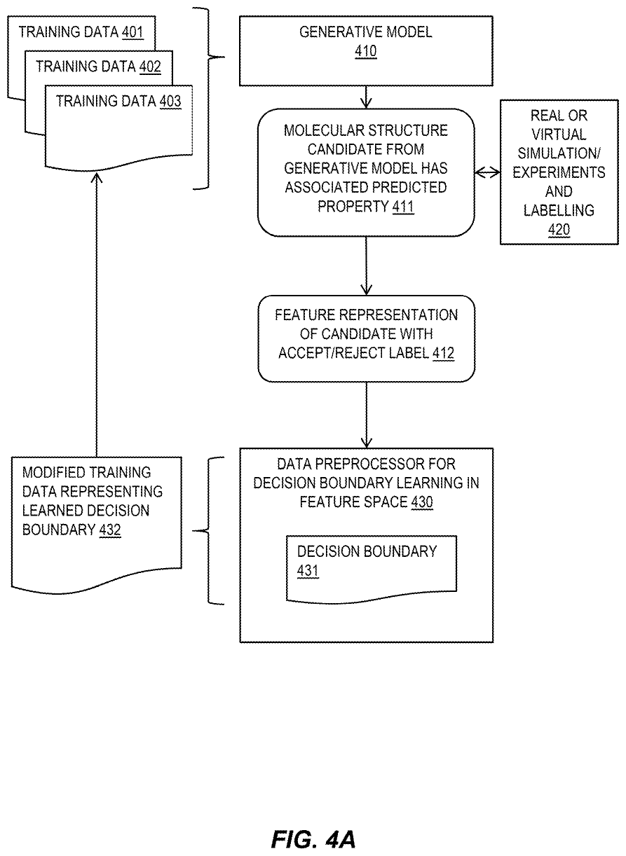

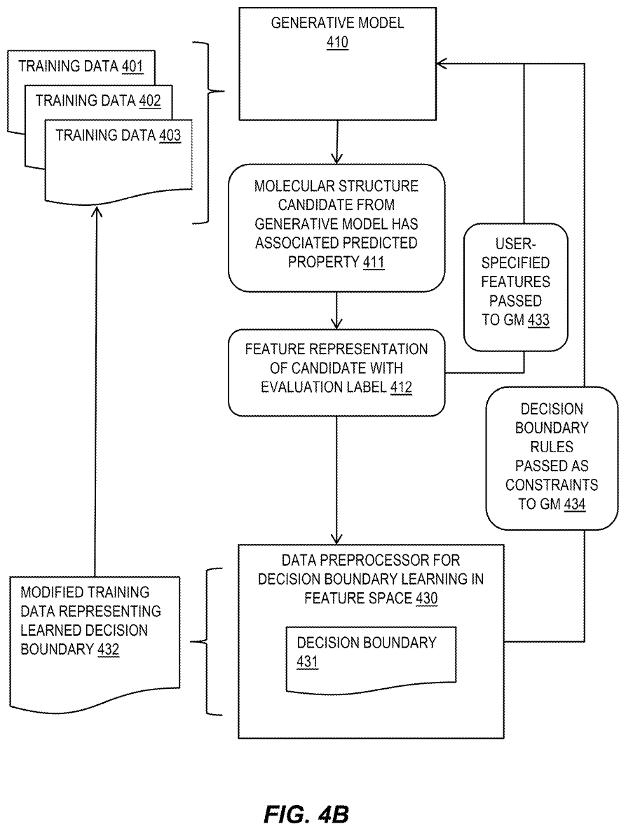

Referring to FIG. 4A, a second embodiment is described using a schematic flow diagram of the method and system components. The second embodiment uses predicted property drift decision boundaries.

The method may input training data 401-403 into a generative model 410. The input data 401-403 may be SMILE string representations and features, e.g., atom counts, substructure counts, InChiKeys (International Chemical Identifiers) or Morgan Fingerprint float features.

The generative model 410 may provide feature encoding, property prediction, feature search, structure generation and may output molecular structure candidates 411 as scaffold candidates with associated predicted property values, e.g. toxicity values. The candidate's actual property is measured 420 via real or virtual simulations or experiments. A threshold tolerance level for the difference between a predicted and actual property value can be used to label candidates with evaluation labels. The labels are instances of predicted property drift tolerance.

Feature representations 412 are provided of the candidates and predicted property drift labels and these are sent to be processed by a data preprocessor 430 to learn the decision boundaries 431 in the feature space.

Modified training data 432 is provided using chemical similarity measures in feature space to represent learned decision boundary, for example, rdkit.DataStructs.FingerprintSimilarity( ).

Referring to FIG. 4B, a third embodiment is described using a schematic flow diagram of the method and system components. The third embodiment includes direct use of constraints in the generative models 310, 410 of the first or second embodiments.

FIG. 4B shows the method and system components of FIG. 4A. In the third embodiment, the features given to data preprocessor are passed as user-specified features 433 to the generative model 410. The rules to determine the decision boundary 434 are passed as constraints of the generative model 410.

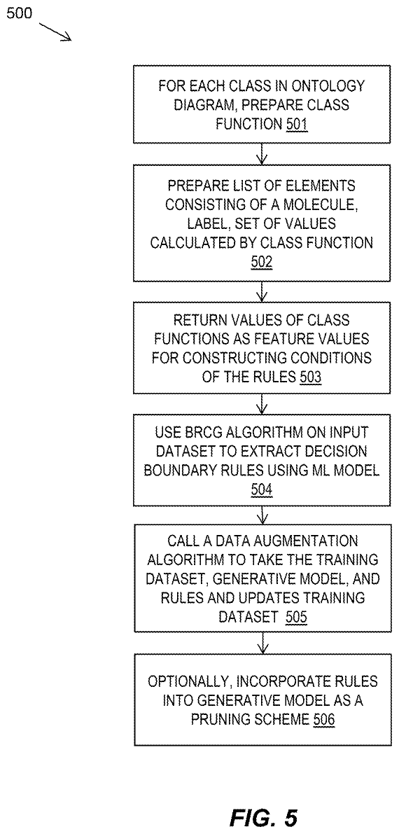

Referring to FIG. 5, a flow diagram 500 shows an example embodiment of the aspect of the described method of the generation of inputs to the data preprocessor for determining decision boundary rules and the data preprocessor functions.

For each class in an ontology diagram, a class function may be prepared 501 which checks whether a molecule in question belongs to that class or not. For example, IsIonicSalts(m), IsAnion(m) IsCation(m), IsOnium(m), etc. These class functions may be embodied as hard-coded implementations (if no labeled data is available) or as machine learning models (if labeled data is available).

The method may prepare 502 a list of elements where each element consists of a molecule, a label (accept/reject) manually annotated by SMEs or automatically annotated by the predicted property drift, and a partial/full set of the values calculated by the class functions. For example,
ID=1, m=“N=[NH2+]”, label=“Accept”, IsIonicSalts(m)=1,IsOnium (m)=1, IsAnion (m)=0 . . .
ID=2, m=“CC=O”, label=“Reject”, IsNonIonOrganic (m)=1

Return 503 values of the class functions as feature values for constructing the conditions of the rules. Generated molecular candidates (a feature vector for each candidate) and candidate labels (e.g., SME labels or labels based on predicted property drift) are provided as input datasets to the data preprocessor component.

A data preprocessor may use 504 a Boolean Rule Column Generation (BRCG) algorithm in order to extract Boolean rules using a machine learning model (for example, logistic regression) and the input dataset.

The BRCG implements a directly interpretable supervised learning method for binary classification that learns a Boolean rule in disjunctive normal form (DNF) or conjunctive normal form (CNF) using column generation (CG). These Boolean rules represent the learned decision boundaries.

Example Rules from BRCG:
IF (752==‘1’) THEN target_class=reject
IF (1814==‘0’ & 35==‘0’ & 449==‘0’) THEN target_class=reject
IF (793==‘1’) THEN target_class=accept
IF (116==‘0’ & 427==‘1’) THEN target_class=accept

A data preprocessor component may call 505 a data augmentation algorithm which takes the training dataset, the generative model, and the Boolean rules, and updates and augments the training dataset according to those rules. Once the generative model is retrained on the new dataset, the output candidates should reflect the learned decision boundaries.

One embodiment for the data augmentation algorithm may be a modified version of solution described in United States Published Patent Application No. US 2022/0358397 “Moving Decision Boundaries of a Machine Learning Model Through Data Manipulation”, which is implemented as a pre-processing algorithm called FROTE (Feedback Rule Based Oversampling Technique). FROTE takes a dataset, a machine-learning model, and user feedback, and it augments the dataset so as to enforce the new boundaries that are enclosed by the user feedback to the machine-learning model. This solution may further be adjusted in the present disclosure to take into account chemical similarity measures for training data augmentation.

When the rules from BRCG are constructed with a CNF/DNF of Boolean formulas regarding feature counts of the second embodiment of generative model, the rules may be incorporated 506 into the generative model as an effective pruning scheme (see the slide later). For example, IF (“# of atom ‘C’>20 or # of substructure ‘—OH’>1”==‘1’) THEN target_class=reject.

The described method has the benefit of using data augmentation to enhance the dataset before retraining generative models. With data augmentation, the solution can enforce the expert feedback into the training dataset that will be input to the generative model. Augmentation will create statistically similar variations to the data through controlling their alignment with the expert decisions.

### Example 1: Generative Model

Input: a training data whose element consists of a molecule represented as SMILES and a target property value (e.g., toxicity score).

Output: At least one pair of a molecule represented as SMILES and its predicted target property value.

One embodiment is to construct a deep neural network based on variational autoencoders to perform the following steps. For example, Gómez-Bombarelli et al. 2018, https://arxiv.org/pdf/1610.02415.pdf.
Learn the latent space of the molecules with the training data;
Perform (biased) samplings in the latent space to find the samples which predict good target property values;
Decode the samplings to molecular graphs/SMILES; and
Return a list whose element consists of a molecule (i.e., SMILES) and its predicted value.

### Example 2-Generative Model

Another embodiment is to combine a machine learning algorithm with a search-based molecular graph construction. For example, Takeda et al. 2020, 2021, https://dl.acm.org/doi/pdf/10.1145/3394486.3403346 https://arxiv.org/pdf/2108.03044.pdf to:

Extract features from the molecule data as internally defined or optionally as specified by the user.
Learn to predict target values with the extracted features (e.g., Support Vector Regression, RandomForest, etc.);
Enumerate molecular graphs which have good predicted values (e.g., value>30.0) by keeping connecting molecular substructures;
Use Molecular Customized Mckay's Canonical Construction Path algorithm;
Convert molecular graphs to SMILES; and
Return a list whose element consists of a molecule (i.e., SMILES) and its predicted value.

In this embodiment, new features defined during the SME's annotation phase can be augmented as user-specified features for improving the accuracy of the target value prediction

### Example 3—Generative Models

Learned decision boundaries may be passed as an optional input for the generative models. One embodiment is to filter out the molecules which are rejected by these boundaries.

If a condition in the if-then-else rule refers to a feature in Example 2 of the generative model, a molecule which is during a construction may be removed immediately when that condition leads to a reject. For example, for rule “if # of C>=10 then reject”, a molecule with more than 10 carbons do not need to be generated without generating all kinds of molecules with # of C>=10).

For other cases, for example, where Example 1 is used as the generative model or the conditions in the rules do not refer to features in Example 2, the molecules rejected by the decision boundaries can be filtered out as follows:
Generate one molecule (completely); and
Check if that molecule is accepted/rejected by following the rules in the decision boundaries.

The described method and system use learned decision boundaries to inform generative models resulting in a targeted, accelerated way to produce molecular candidates. The learned decision boundaries calculated in this method may be used to limit the feature space under consideration and target areas in feature space of high relevance, thus improving the efficiency of molecule generation this may be used for chemical applications such as material discovery.

Discovery accelerator platforms are composed of modules, such as generative models, expert-in-the-loop triage and virtual experiments or simulations. The proposed system may represent an intelligent infrastructure component of such a platform capable of combining these modules, learning from the outputs, and increasing the efficiency of molecular generation.

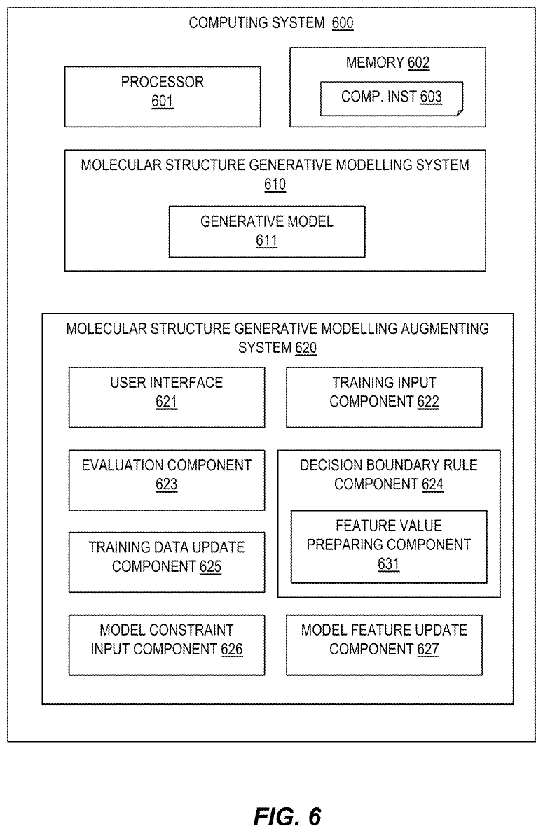

FIG. 6 shows a block diagram of a computing system 600 in which the described system may be implemented. The computing system 600 includes at least one processor 601, a hardware module, or a circuit for executing the functions of the described components which may be software units executing on the at least one processor. Multiple processors running parallel processing threads may be provided enabling parallel processing of some or all of the functions of the components. Memory 602 may be configured to provide computer instructions 603 to the at least one processor 601 to carry out the functionality of the components.

The described system is provided in association with a molecular structure generative modelling system 610 having a generative model 611. The described system is a molecular structure generative model augmenting system 620 including a user interface 621 or interaction between a user and the generative modelling system 610. The molecular structure generative model augmenting system may be incorporated into a molecular discovery accelerator platform including the generative modelling system 610.

The molecular structure generative model augmenting system 620 includes a training input component 622 for providing labelled training data for training the generative model 611 over a defined feature space. The labelled training data including representations of molecular structure and property values for each molecular structure to output generated molecular structures with target properties.

The model augmenting system 620 includes an evaluation component 623 for receiving evaluations of the generated candidate molecular structure outputs to provide feature representations of candidates with evaluation labels. In one embodiment, the evaluation component 623 receives evaluations of the generated molecular structure outputs receives evaluations from a subject matter expert using ontological feature representations including evaluation labels.

In another embodiment, the evaluation component 624 receives evaluations of the generated molecular structure outputs in the form of predicted property drift labels obtained by measuring predicted property values against tested property values. The evaluation component 624 may receive evaluations of the generated molecular structure outputs using previously generated decision boundary rules when available.

The model augmenting system 620 includes a decision boundary rule component 624 for generating or updating decision boundary rules based on the evaluations. The decision boundary rule component 624 may include a feature value preparing component 631 for preparing feature values for constructing conditions for the decision boundary rules including: preparing a class function for each class in an ontology diagram, where the class function checks whether a molecule in question belongs to that class or not; and preparing a list of elements where each element consists of a molecule, a label, and a set of the values calculated by the class functions.

The model augmenting system 620 includes a training data update component 625 for applying the decision boundary rules to update the labelled training data. The training data update component 625 may be for modifying training data using chemical similarity measures in the feature space to represent learned decision boundaries.

The model augmenting system 620 may include a model constraint input component 626 for modifying a generation algorithm of the generative model with structural constraints representing the decision boundary rules.

The model augmenting system 620 may include a model feature update component 627 for passing features of feature representations of candidates with evaluation labels to generative model as user-specified features to update the generative model.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

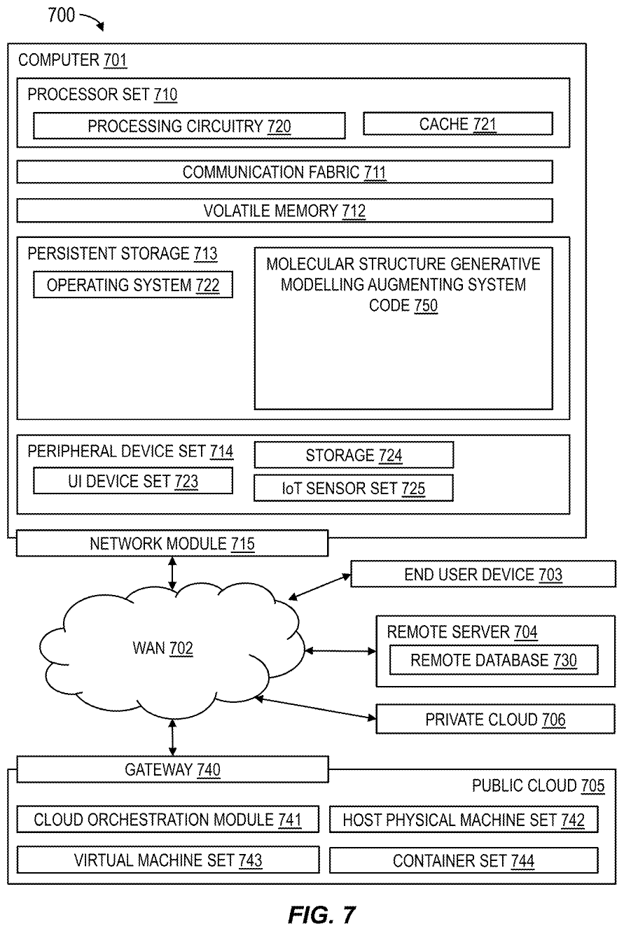

Referring to FIG. 7, computing environment 700 contains an example of an environment for the execution of at least some of the computer code involved in performing the inventive methods, such as molecular structure generative modelling augmenting system code 750. In addition to block 750, computing environment 700 includes, for example, computer 701, wide area network (WAN) 702, end user device (EUD) 703, remote server 704, public cloud 705, and private cloud 706. In this embodiment, computer 701 includes processor set 710 (including processing circuitry 720 and cache 721), communication fabric 711, volatile memory 712, persistent storage 713 (including operating system 722 and block 750, as identified above), peripheral device set 714 (including user interface (UI) device set 723, storage 724, and Internet of Things (IoT) sensor set 725), and network module 715. Remote server 704 includes remote database 730. Public cloud 705 includes gateway 740, cloud orchestration module 741, host physical machine set 742, virtual machine set 743, and container set 744.

COMPUTER 701 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 730. As is well understood in the art of computer technology, and depending upon the technology, performance of a computer-implemented method may be distributed among multiple computers and/or between multiple locations. On the other hand, in this presentation of computing environment 700, detailed discussion is focused on a single computer, specifically computer 701, to keep the presentation as simple as possible. Computer 701 may be located in a cloud, even though it is not shown in a cloud in FIG. 7. On the other hand, computer 701 is not required to be in a cloud except to any extent as may be affirmatively indicated.

PROCESSOR SET 710 includes one, or more, computer processors of any type now known or to be developed in the future. Processing circuitry 720 may be distributed over multiple packages, for example, multiple, coordinated integrated circuit chips. Processing circuitry 720 may implement multiple processor threads and/or multiple processor cores. Cache 721 is memory that is located in the processor chip package(s) and is typically used for data or code that should be available for rapid access by the threads or cores running on processor set 710. Cache memories are typically organized into multiple levels depending upon relative proximity to the processing circuitry. Alternatively, some, or all, of the cache for the processor set may be located “off chip.” In some computing environments, processor set 710 may be designed for working with qubits and performing quantum computing.

Computer readable program instructions are typically loaded onto computer 701 to cause a series of operational steps to be performed by processor set 710 of computer 701 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 721 and the other storage media discussed below. The program instructions, and associated data, are accessed by processor set 710 to control and direct performance of the inventive methods. In computing environment 700, at least some of the instructions for performing the inventive methods may be stored in block 750 in persistent storage 713.

COMMUNICATION FABRIC 711 is the signal conduction path that allows the various components of computer 701 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 712 is any type of volatile memory now known or to be developed in the future. Examples include dynamic type random access memory (RAM) or static type RAM. Typically, volatile memory 712 is characterized by random access, but this is not required unless affirmatively indicated. In computer 701, the volatile memory 712 is located in a single package and is internal to computer 701, but, alternatively or additionally, the volatile memory may be distributed over multiple packages and/or located externally with respect to computer 701.

PERSISTENT STORAGE 713 is any form of non-volatile storage for computers that is now known or to be developed in the future. The non-volatility of this storage means that the stored data is maintained regardless of whether power is being supplied to computer 701 and/or directly to persistent storage 713. Persistent storage 713 may be a read only memory (ROM), but typically at least a portion of the persistent storage allows writing of data, deletion of data and re-writing of data. Some familiar forms of persistent storage include magnetic disks and solid state storage devices. Operating system 722 may take several forms, such as various known proprietary operating systems or open source Portable Operating System Interface-type operating systems that employ a kernel. The code included in block 750 typically includes at least some of the computer code involved in performing the inventive methods.

PERIPHERAL DEVICE SET 714 includes the set of peripheral devices of computer 701. Data communication connections between the peripheral devices and the other components of computer 701 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 723 may include components such as a display screen, speaker, microphone, wearable devices (such as goggles and smart watches), keyboard, mouse, printer, touchpad, game controllers, and haptic devices. Storage 724 is external storage, such as an external hard drive, or insertable storage, such as an SD card. Storage 724 may be persistent and/or volatile. In some embodiments, storage 724 may take the form of a quantum computing storage device for storing data in the form of qubits. In embodiments where computer 701 is required to have a large amount of storage (for example, where computer 701 locally stores and manages a large database) then this storage may be provided by peripheral storage devices designed for storing very large amounts of data, such as a storage area network (SAN) that is shared by multiple, geographically distributed computers. IoT sensor set 725 is made up of sensors that can be used in Internet of Things applications. For example, one sensor may be a thermometer and another sensor may be a motion detector.

NETWORK MODULE 715 is the collection of computer software, hardware, and firmware that allows computer 701 to communicate with other computers through WAN 702. Network module 715 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 715 are performed on the same physical hardware device. In other embodiments (for example, embodiments that utilize software-defined networking (SDN)), the control functions and the forwarding functions of network module 715 are performed on physically separate devices, such that the control functions manage several different network hardware devices. Computer readable program instructions for performing the inventive methods can typically be downloaded to computer 701 from an external computer or external storage device through a network adapter card or network interface included in network module 715.

WAN 702 is any wide area network (for example, the internet) capable of communicating computer data over non-local distances by any technology for communicating computer data, now known or to be developed in the future. In some embodiments, the WAN 702 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 703 is any computer system that is used and controlled by an end user (for example, a customer of an enterprise that operates computer 701), and may take any of the forms discussed above in connection with computer 701. EUD 703 typically receives helpful and useful data from the operations of computer 701. For example, in a hypothetical case where computer 701 is designed to provide a recommendation to an end user, this recommendation would typically be communicated from network module 715 of computer 701 through WAN 702 to EUD 703. In this way, EUD 703 can display, or otherwise present, the recommendation to an end user. In some embodiments, EUD 703 may be a client device, such as thin client, heavy client, mainframe computer, desktop computer and so on.

REMOTE SERVER 704 is any computer system that serves at least some data and/or functionality to computer 701. Remote server 704 may be controlled and used by the same entity that operates computer 701. Remote server 704 represents the machine(s) that collect and store helpful and useful data for use by other computers, such as computer 701. For example, in a hypothetical case where computer 701 is designed and programmed to provide a recommendation based on historical data, then this historical data may be provided to computer 701 from remote database 730 of remote server 704.

PUBLIC CLOUD 705 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 705 is performed by the computer hardware and/or software of cloud orchestration module 741. The computing resources provided by public cloud 705 are typically implemented by virtual computing environments that run on various computers making up the computers of host physical machine set 742, which is the universe of physical computers in and/or available to public cloud 705. The virtual computing environments (VCEs) typically take the form of virtual machines from virtual machine set 743 and/or containers from container set 744. It is understood that these VCEs may be stored as images and may be transferred among and between the various physical machine hosts, either as images or after instantiation of the VCE. Cloud orchestration module 741 manages the transfer and storage of images, deploys new instantiations of VCEs and manages active instantiations of VCE deployments. Gateway 740 is the collection of computer software, hardware, and firmware that allows public cloud 705 to communicate through WAN 702.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 706 is similar to public cloud 705, except that the computing resources are only available for use by a single enterprise. While private cloud 706 is depicted as being in communication with WAN 702, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 705 and private cloud 706 are both part of a larger hybrid cloud.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Improvements and modifications can be made to the foregoing without departing from the scope of the present invention.

What is claimed is:

1. A computer-implemented method for generative modelling of molecular structures for chemical applications, the method comprising:
providing labelled training data for training a generative model over a defined feature space, wherein the labelled training data includes representations of molecular structures and property values for each molecular structure, and wherein the generative model outputs generated candidate molecular structures with target properties;
receiving evaluations of generated candidate molecular structure outputs from the generative model, wherein the evaluations provide feature representations of candidates with evaluation labels;
generating or updating decision boundary rules based on the evaluations; and
applying the decision boundary rules to update the labelled training data.

2. The method of claim 1, further comprising:
modifying a generation algorithm of the generative model with structural constraints representing the decision boundary rules.

3. The method of claim 1, further comprising:
passing features of feature representations of candidates with evaluation labels to generative model as user-specified features to update the generative model.

4. The method of claim 1, further comprising:
modifying training data using chemical similarity measures in the feature space to represent learned decision boundaries.

5. The method of claim 1, wherein generating decision boundary rules includes preparing feature values for constructing conditions for the decision boundary rules including:
preparing a class function for each class in an ontology diagram, where the class function checks whether a molecule in question belongs to that class or not; and
preparing a list of elements where each element consists of a molecule, a label, and a set of the values calculated by the class functions.

6. The method of claim 1, wherein receiving evaluations of the generated molecular structure outputs receives evaluations from a subject matter expert using ontological feature representations to provide evaluation labels of candidate representations.

7. The method of claim 1, wherein receiving evaluations of the generated molecular structure outputs comprises: measuring predicted property values against tested property values to provide evaluation labels of candidate representations in the form of predicted property drift labels.

8. The method of claim 7, wherein tested property values are obtained by real or simulated experimental data.

9. The method of claim 1, wherein receiving evaluations of the generated molecular structure outputs receives evaluations using previously generated decision boundary rules.

10. A computer system for generative modelling of molecular structures for chemical applications, comprising:
one or more processors;
a memory coupled to at least one of the processors;
a set of computer program instructions stored in the memory and executed by at least one of the processors in order to perform actions of:
providing, by a training input component, labelled training data for training a generative model over a defined feature space, wherein the labelled training data includes representations of molecular structures and property values for each molecular structure, and wherein the generative model outputs generated candidate molecular structures with target properties;
receiving, by an evaluation component, evaluations of generated candidate molecular structure outputs from the generative model, wherein the evaluations provide feature representations of candidates with evaluation labels;
generating, by a decision boundary rule component, decision boundary rules based on the evaluations; and
applying, by a training update component, the decision boundary rules to update the labelled training data.

11. The computer system of claim 10, including:
modifying, by a model constraint input component, a generation algorithm of the generative model with structural constraints representing the decision boundary rules.

12. The computer system of claim 10, comprising:
passing, by a model feature update component, features of feature representations of candidates with evaluation labels to generative model as user-specified features to update the generative model.

13. The computer system of claim 10, wherein the training data update component modifies training data using chemical similarity measures in the feature space to represent learned decision boundaries.

14. The computer system of claim 10, wherein the decision boundary rule component includes a feature value preparing component for preparing feature values for constructing conditions for the decision boundary rules including:
preparing a class function for each class in an ontology diagram, where the class function checks whether a molecule in question belongs to that class or not; and
preparing a list of elements where each element consists of a molecule, a label, and a set of the values calculated by the class functions.

15. The computer system of claim 10, wherein the evaluation component receives evaluations of the generated molecular structure outputs receives evaluations from a subject matter expert using ontological feature representations to provide evaluation labels of candidate representations.

16. The computer system of claim 10, wherein the evaluation component receives evaluations of the generated molecular structure outputs in the form of predicted property drift labels obtained by measuring predicted property values against tested property values.

17. The computer system of claim 10, wherein the evaluation component receives evaluations of the generated molecular structure outputs using previously generated decision boundary rules when available.

18. The computer system of claim 10, further comprising a user interface for interaction between the user and the modelling system for providing evaluation labels.

19. The computer system of claim 10, wherein the system is incorporated into a molecular discovery accelerator platform including a generative model.

20. A computer program product, the computer program product comprising a non-transitory tangible storage device having program code embodied therewith, the program code executable by a processor of a computer to perform a method, the method comprising:
providing labelled training data for training a generative model over a defined feature space, wherein the labelled training data includes representations of molecular structures and property values for each molecular structure, and wherein the generative model outputs generated candidate molecular structures with target properties;
receiving evaluations of generated candidate molecular structure outputs from the generative model, wherein the evaluations provide feature representations of candidates with evaluation labels;
generating or updating decision boundary rules based on the evaluations; and
applying the decision boundary rules to update the labelled training data.

21. A computer system for molecular structure generative model augmenting, comprising:
one or more processors;
a memory coupled to at least one of the processors;
a set of computer program instructions stored in the memory and executed by at least one of the processors in order to perform actions of:
providing, by a training input component, labelled training data for training a generative model over a defined feature space, wherein the labelled training data includes representations of molecular structures and property values for each molecular structure, and wherein the generative model outputs generated candidate molecular structures with target properties;
receiving, by an evaluation component, evaluations of generated candidate molecular structure outputs from the generative model, wherein the evaluations provide feature representations of candidates with evaluation labels;
generating, by a decision boundary rule component, decision boundary rules based on the evaluations; and
applying, by a training update component, the decision boundary rules to update the labelled training data.

22. A computer-implemented method for molecular structure generative model augmenting, the method comprising:
providing labelled training data for training a generative model over a defined feature space, wherein the labelled training data includes representations of molecular structures and property values for each molecular structure, and wherein the generative model outputs generated candidate molecular structures with target properties;
receiving evaluations of generated candidate molecular structure outputs from the generative model, wherein the evaluations provide feature representations of candidates with evaluation labels;
generating or updating decision boundary rules based on the evaluations;
applying the decision boundary rules to update the labelled training data, wherein the training data is updated using chemical similarity measures in the feature space to represent learned decision boundaries;
modifying, by a model constraint input component, a generation algorithm of the generative model with structural constraints representing the decision boundary rules; and
passing, by a model feature update component, features of feature representations of candidates with evaluation labels to generative model as user-specified features to update the generative model.
